(19) United States
(12) Patent Application Publication
(10) Pub. No.: US 2024/0273899 A1
(43) Pub. Date: Aug. 15, 2024

(54) AI-Powered Surgical Video Analysis

(71) Applicant: REGENTS OF THE UNIVERSITY OF MICHIGAN, Ann Arbor, MI (US)

(72) Inventors: Nambi Nallasamy, Ann Arbor, MI (US); Bradford Tannen, Superior Township, MI (US); Shahzad Mian, Superior Township, MI (US)

(21) Appl. No.: 18/440,436

(22) Filed: Feb. 13, 2024

Related U.S. Application Data
(60) Provisional application No. 63/445,053, filed on Feb. 13, 2023.

(51) Int. Cl. G06V 20/40; G06Q 10/0639; G06T 7/00; G06V 10/26; G06V 10/70; G16H 40/20

(52) U.S. Cl. CPC G06V 20/41; G06Q 10/06398; G06T 7/0012; G06V 10/273; G06V 10/70; G16H 40/20; G06T 2207/10016; G06T 2207/20081; G06T 2207/30041; G06V 2201/034

(57) ABSTRACT

Systems and methods for using machine learning to analyze a surgical video and assess performance of a surgeon conducting a surgical procedure which may include receiving surgical video data including one or more images capturing at least a portion of an ophthalmic surgical procedure from a user device. Processing the surgical video data using one or more trained assessment machine learning models to generate one or more assessment metrics, wherein the one or more trained assessment machine learning models are trained using historical ophthalmic surgery data and the one or more assessment metrics include one or more of a surgical instrument metric, a surgical phase metric, or an anterior capsulotomy metric. Generating a performance assessment of the surgeon based upon at least the one or more assessment metrics and providing the performance assessment of the surgeon to the user device.

### GOVERNMENT LICENSE RIGHTS

This invention was made with government support under EY022299 awarded by the National Institutes of Health (NIH). The government has certain rights in the invention.

### FIELD OF THE DISCLOSURE

The present disclosure is generally directed to techniques for using machine learning to analyze a surgical video to assess performance of a surgeon conducting a surgical procedure.

### BACKGROUND

Presently, providing an assessment of surgical performance is an exceedingly manual, subjective, and imperfect process. One or more persons may be tasked with reviewing a surgery to provide feedback and the assessment they provide may be susceptible to any number of detrimental defects, for example: the reviewer may be impartial due to familiarity with the subject; the reviewer may lack the requisite expertise to provide a quality, informed assessment; the reviewer may only be able to provide feedback in their “downtime” and/or during off-hours, leading to reviewer burnout and/or rushing through the feedback process; and/or inconsistency between reviews may arise due to subjectivity, which may be difficult, if not impossible, to avoid.

Additionally, the feedback itself may be of limited use. As assessment that would be helpful, if not essential, to providing an improved surgical outcome may be received after a surgery is finished to avoid interrupting the surgeon and/or concerning the patient. Moreover, there may not be enough and/or consistent feedback over time for a surgeon to get a sense of the trajectory of their skill.

Therefore, there is an opportunity for providing an improved assessment of surgical performance which is automated, timely, and objective to avoid the shortcomings and inconsistencies of the present techniques.

### BRIEF SUMMARY

In one aspect, a computer-implemented method of using machine learning for analyzing a surgical video to assess performance of a surgeon conducting a surgical procedure. The method may include receiving, by one or more processors, surgical video data including one or more images capturing at least a portion of an ophthalmic surgical procedure; processing, by the one or more processors, the surgical video data using one or more trained assessment machine learning models to generate one or more assessment metrics, wherein: the one or more trained assessment machine learning models are trained using historical ophthalmic surgery data; and the one or more assessment metrics include one or more of a surgical instrument metric, a surgical phase metric, or an anterior capsulotomy metric; generating, by the one or more processors and based upon at least the one or more assessment metrics, a performance assessment of the surgeon; and providing, by the one or more processors to a user device, the performance assessment of the surgeon.

In another aspect, a computer system for using machine learning to analyze a surgical video and assess performance of a surgeon conducting a surgical procedure. The computer system may include one or more processors and a memory comprising instructions, that when executed, cause the computer system to: receive surgical video data including one or more images capturing at least a portion of an ophthalmic surgical procedure from a user device; process the surgical video data using one or more trained assessment machine learning models to generate one or more assessment metrics, wherein: the one or more trained assessment machine learning models are trained using historical ophthalmic surgery data; and the one or more assessment metrics include one or more of a surgical instrument metric, a surgical phase metric, or an anterior capsulotomy metric; generate a performance assessment of the surgeon based upon at least the one or more assessment metrics; and provide the performance assessment of the surgeon to the user device.

In yet another aspect, a non-transitory computer-readable storage medium storing executable instructions that, when executed by a processor, cause a computer to analyze a surgical video and assess performance of a surgeon conducting a surgical procedure. In an aspect, the computer may receive surgical video data including one or more images capturing at least a portion of an ophthalmic surgical procedure from a user device; process the surgical video data using one or more trained assessment machine learning models to generate one or more assessment metrics, wherein: the one or more trained assessment machine learning models are trained using historical ophthalmic surgery data; and the one or more assessment metrics include one or more of a surgical instrument metric, a surgical phase metric, or an anterior capsulotomy metric; generate a performance assessment of the surgeon based upon at least the one or more assessment metrics; and provide the performance assessment of the surgeon to the user device.

### BRIEF DESCRIPTION OF THE DRAWINGS

The figures described below depict various aspects of the system and methods disclosed therein. It should be understood that each figure depicts one embodiment of a particular aspect of the disclosed system and methods, and that each of the figures is intended to accord with a possible embodiment thereof. Further, wherever possible, the following description refers to the reference numerals included in the following figures, in which features depicted in multiple figures are designated with consistent reference numerals.

There are shown in the drawings arrangements which are presently discussed, it being understood, however, that the present aspects are not limited to the precise arrangements and instrumentalities shown, wherein:

FIG. 1 depicts a computing environment in which machine learning may analyze a surgical video and assess performance of a surgeon conducting a surgical procedure, in some aspects;

FIG. 2 depicts an exemplary block diagram depicting a high-level system flow for using machine learning to analyze a surgical video and assess performance of a surgeon, according to an aspect;

FIG. 3 depicts an exemplary image in which machine learning identifies surgical instruments in image data, according to an aspect;

FIG. 4 depicts an exemplary block diagram in which machine learning generates edited surgical video data removing phases of inactivity; according to an aspect;

FIG. 5 depicts an exemplary block diagram in which machine learning generates semantically segmented capsulorrhexis image data, according to an aspect;

FIG. 6 depicts an exemplary block flow diagram depicting a computer-implemented method for using machine learning to analyze a surgical video and assess performance of a surgeon, according to an aspect.

### DETAILED DESCRIPTION

### Overview

The aspects described herein relate to, inter alia, using machine learning (ML) to analyze a surgical video and assess performance of a surgeon conducting a surgical procedure.

Specifically, the present techniques include methods and systems for receiving by a surgical video analysis and assessment system (SVAAS) surgical video data including one or more images capturing at least a portion of an ophthalmic surgical procedure. The SVAAS may process the surgical video data using one or more trained assessment machine learning models to generate one or more assessment metrics, and may generate and provide to a user device a performance assessment of the surgeon based upon at least the one or more assessment metrics.

The SVAAS may also include a trained machine learning model to edit the surgical video data to remove images capturing phase inactivity and may also include a trained machine learning model to generate from the surgical video data, a semantic segmentation subset data including at least one or more images of a classified capsulorrhexis.

### Exemplary Computing Environment

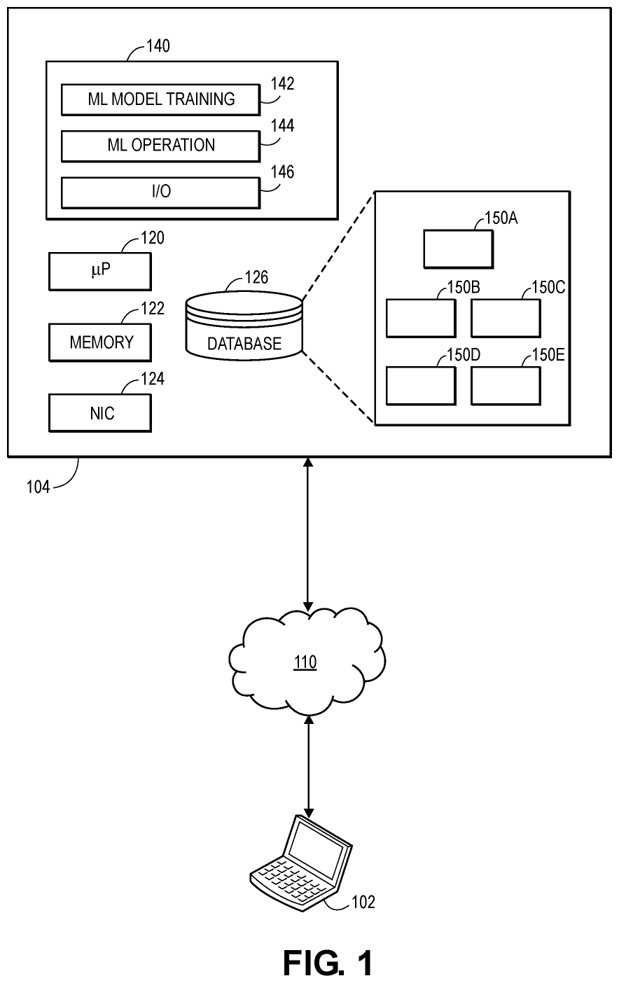

FIG. 1 depicts a computing environment 100 in which machine learning (ML) may analyze a surgical video and assess performance of a surgeon conducting a surgical procedure, in accordance with various aspects discussed herein.

In the example aspect of FIG. 1, computing environment 100 includes user device 102. In various aspects, user device 102 may comprise a single computer, multiple computers which may comprise multiple, redundant, or replicated client computers accessed by one or more users. The environment 100 may further include an electronic network 110 communicatively coupling other aspects of the environment 100.

The user device 102 may be any suitable device (e.g., a laptop, a smartphone, a tablet, a wearable device, a blade server, etc.). The user device 102 may include a user interface (e.g., a display, touchscreen, keyboard, and/or mouse), as well as a memory and a processor for, respectively, storing and executing one or more modules. The memory may include one or more suitable storage media such as a magnetic storage device, a solid-state drive, random access memory (RAM), etc. The user device 102 may access services or other components of the environment 100 via the network 110. In an example, the user device 102 may access the SVAAS 104 via network 110, allowing the user device 102 to interact with the SVAAS 104 in various ways, including but not limited to providing a surgical video data to the SVAAS 104, selecting one or more metrics for the SVAAS 104 to generate from the surgical video data, selecting one or more performance assessments for the SVAAS 104 to generate from the assessment metrics and/or surgical video data, and/or otherwise interact with, interface with, and/or control the SVAAS 104.

As described herein and in some aspects, the SVAAS 104 may perform functionalities as part of a network or may otherwise communicate with other hardware or software components within one or more computing environments to send, retrieve, process, and/or otherwise analyze data or information described herein. For example, in aspects of the present techniques, the computing environment 100 may comprise an on-premise computing environment, a multi-cloud computing environment, a public cloud computing environment, a private cloud computing environment, and/or a hybrid cloud computing environment. For example, any entity (e.g., a business or academic institution) offering surgical video analysis and assessment of a surgeon may host one or more services in a public cloud computing environment (e.g., Alibaba Cloud, Amazon Web Services (AWS), Google Cloud, IBM Cloud, Microsoft Azure, etc.). The public cloud computing environment may be a traditional off-premise cloud (i.e., not physically hosted at a location owned/controlled by the business). Alternatively, or in addition, aspects of the public cloud may be hosted on-premise at a location owned/controlled by the business and/or academic institution offering the SVAAS 104. The public cloud may be partitioned using visualization and multi-tenancy techniques and may include one or more infrastructure-as-a-service (IaaS) and/or platform-as-a-service (PaaS) services.

The network 110 may comprise any suitable network or networks, including a local area network (LAN), wide area network (WAN), Internet, or combination thereof. For example, the network 110 may include a wireless cellular service (e.g., 4G, 5G, etc.). Generally, the network 110 enables bidirectional communication between the user device 102 and the SVAAS 104. In some aspects, network 110 may comprise a cellular base station, such as cell tower(s), communicating to the one or more components of the environment 100 via wired/wireless communications based on any one or more of various mobile phone standards, including NMT, GSM, CDMA, UMMTS, LTE, 5G, or the like. Additionally, or alternatively, network 110 may comprise one or more routers, wireless switches, or other such wireless connection points communicating to the components of the environment 100 via wireless communications based on any one or more of various wireless standards, including by non-limiting example, IEEE 802.11a/b/c/g (WIFI), Bluetooth, and/or the like.

The SVAAS 104 may include one or more processors 120, one or more computer memories 122, one or more network interface controllers (NICs) 124 and one or more electronic databases 126. The NIC 124 may include any suitable network interface controller(s) and may communicate over the network 110 via any suitable wired and/or wireless connection. The SVAAS 104 may include one or more input devices (not depicted) and may include one or more devices for allowing a user to enter inputs (e.g., data) into the SVAAS 104. For example, the input device may include a keyboard, a mouse, a microphone, a camera, etc. The NIC may include one or more transceivers (e.g., WWAN, WLAN, and/or WPAN transceivers) functioning in accordance with IEEE standards, 3GPP standards, or other standards, and that may be used in receipt and transmission of data via external/network ports connected to computer network 110.

The processor 120 may include one or more suitable processors (e.g., central processing units (CPUs) and/or graphics processing units (GPUs)). The processor 120 may be connected to the memory 122 via a computer bus (not depicted) responsible for transmitting electronic data, data packets, or otherwise electronic signals to and from the processor 120 and memory 122 in order to implement or perform the machine-readable instructions, methods, processes, elements or limitations, as illustrated, depicted, or described for the various flowcharts, illustrations, diagrams, figures, and/or other disclosure herein. The processor 120 may interface with the memory 122 via a computer bus to execute an operating system (OS) and/or computing instructions contained therein, and/or to access other services/aspects. For example, the processor 120 may interface with the memory 122 via the computer bus to create, read, update, delete, or otherwise access or interact with the data stored in memory 122 and/or the database 126.

The memory 122 may include one or more forms of volatile and/or non-volatile, fixed and/or removable memory, such as read-only memory (ROM), electronic programmable read-only memory (EPROM), random access memory (RAM), erasable electronic programmable read-only memory (EEPROM), and/or other hard drives, flash memory, MicroSD cards, and others. The memory 122 may store an operating system (OS) (e.g., Microsoft Windows, Linux, UNIX, etc.) capable of facilitating the functionalities, apps, methods, or other software as discussed herein.

The memory 122 and/or database 126 may store one or more computing modules 140, implemented as respective sets of computer-executable instructions (e.g., one or more source code libraries, trained ML models such as neural networks, convolutional neural networks, etc.) as described herein.

The memory 122 and/or database 126 may also store data provided by the user, such as surgical video data. The data may include, but is not limited to, a collection of information that is composed of separate elements but may be manipulated as a unit by a computer, processor 120, or the like. The data may be related (e.g., data related to a single surgery) or unrelated, and may only have one element in some circumstances or may include one or more subset data. The term data set, data and subset data may be used interchangeably herein. In an example, a user may upload surgical video data from the user device 102 to the SVAAS 104, and the SVAAS 104 may store the surgical video data in memory 122 and/or database 126, e.g., for further processing by the SVAAS 104 and/or one or more trained ML models.

In general, a computer program or computer based product, application, or code (e.g., one or more models, such as ML models, or other computing instructions described herein) may be stored on a computer usable storage medium, or tangible, non-transitory computer-readable medium (e.g., standard random access memory (RAM), an optical disc, a universal serial bus (USB) drive, or the like) having such computer-readable program code or computer instructions embodied therein, wherein the computer-readable program code or computer instructions may be installed on or otherwise adapted to be executed by the processor(s) 120 (e.g., working in connection with the respective operating system in memory 122) to facilitate, implement, or perform the machine readable instructions, methods, processes, elements or limitations, as illustrated, depicted, or described for the various flowcharts, illustrations, diagrams, figures, and/or other disclosure herein. In this regard, the program code may be implemented in any desired program language, and may be implemented as machine code, assembly code, byte code, interpretable source code or the like (e.g., via Golang, Python, C, C++, C#, Objective-C, Java, Scala, ActionScript, JavaScript, HTML, CSS, XML, etc.).

The database 126 may be a relational database, such as Oracle, DB2, MySQL, a NoSQL based database, such as MongoDB, or another suitable database. The database 126 may store data and be used to train and/or operate one or more ML and/or artificial intelligence (“AI”) models.

The ML training module 142 may receive labeled data (e.g., via memory 122 or database 126) at an input layer of a model having a networked layer architecture (e.g., an artificial neural network, a convolutional neural network, etc.) for training the one or more ML models, such as ML models 150A, 150B, 150C, 150D, 150E. The received data may be propagated through one or more connected deep layers of the ML model to establish weights of one or more nodes, or neurons, of the respective layers. Initially, the weights may be initialized to random values, and one or more suitable activation functions may be chosen for the training process. The present techniques may include training a respective output layer of the one or more ML models. The output layer may be trained to output a prediction, for example.

In various aspects, the ML models as described herein, may be trained using a supervised or unsupervised ML program or algorithm. The ML program or algorithm may employ a neural network, which may be a convolutional neural network, a deep learning neural network, or a combined learning module or program that learns in two or more features or feature data sets (e.g., structured data, unstructured data, etc.) in a particular area of interest. The ML programs or algorithms may include natural language processing (NLP), semantic analysis, automatic reasoning, regression analysis, support vector machine (SVM) analysis, decision tree analysis, random forest analysis, K-Nearest neighbor analysis, naïve Bayes analysis, clustering, reinforcement learning, and/or other ML algorithms and/or techniques. In some aspects, the ML based algorithms may be included as a library or package executed on SVAAS 104. For example, libraries may include the TensorFlow based library, the Pytorch library, and/or the scikit-learn Python library.

ML may involve identifying and recognizing patterns in existing data (e.g., surgical video data or historical ophthalmic surgery data) in order to facilitate making predictions, classifications, and/or identifications for subsequent data (e.g., using models to determine various assessment metrics associated with a surgeon and/or surgery).

ML models may be created and trained based upon example data (e.g., training data) inputs or data (which may be termed “features” and “labels”) in order to make valid and reliable predictions for new inputs. In supervised ML, an ML program operating on a server, computing device, or other processor(s), may be provided with example inputs (e.g., features) and their associated, or observed, outputs (e.g., labels) in order for the ML program or algorithm to determine or discover rules, relationships, patterns that map such inputs (e.g., features) to the outputs (e.g., labels), for example, by determining and/or assigning weights or other metrics to the model across its various feature categories. The process of creating and training ML models may result in models that are digital objects that may be stored in memory or a database and used later after training to make accurate predictions. Such models and the rules/relationships they encode may be provided subsequent inputs in order for the model, executing on the server, computing device, or other processor(s), to predict, based on the discovered rules, relationships, or model, an expected output.

In unsupervised ML, the server, computing device, or otherwise processor(s), may find its own structure in unlabeled example inputs, where, for example multiple training iterations are executed by the server, computing device, or otherwise processor(s) to train multiple generations of models until a satisfactory model, e.g., a model that provides sufficient prediction accuracy when given test level or production level data or inputs, is generated.

Supervised learning and/or unsupervised ML may also include retraining, relearning, or otherwise updating models with new, or different, information, which may include information received, ingested, generated, or otherwise used over time. The disclosures herein may use one or both of such supervised or unsupervised ML techniques.

In some aspects, the computing module 140 may include ML operation module 144, comprising a set of computer-executable instructions implementing ML loading, configuration, initialization and/or operation functionality. The ML operation module 144 may include instructions for storing trained models (e.g., ML models 150A, 150B, 150C, 150D, 150E stored in the database 126). As discussed, once trained, the one or more trained ML models may be operated in inference mode, whereupon when provided with de novo input that the model has not previously been provided, the model may output one or more predictions, classifications, etc., as described herein.

In some aspects, the computing module 140 may include an input/output (I/O) module 146, comprising a set of computer-executable instructions implementing communication functions. The I/O module 146 may include a communication component configured to communicate (e.g., send and receive) data via one or more external/network port(s) to one or more networks or local terminals, such as computer network 110 and/or the user device 102 (for rendering or visualizing) described herein. In some aspects, SVAAS 104 may include a client-server platform technology such as ASP.NET, Java J2EE, Ruby on Rails, Node.js, a web service or online API, responsive for receiving and responding to electronic requests.

I/O module 146 may further include or implement an operator interface configured to present information to an administrator or operator and/or receive inputs from the administrator and/or operator. An operator interface may provide a display screen. I/O module 146 may facilitate I/O components (e.g., ports, capacitive or resistive touch sensitive input panels, keys, buttons, lights, LEDs), which may be directly accessible via, or attached to, SVAAS 104 or may be indirectly accessible via or attached to the user device 102. According to some aspects, an administrator or operator may access the SVAAS 104 via the user device 102 to review/receive/transmit information and/or data, make changes, input training data, initiate training via the ML model training module 142, and/or perform other functions (e.g., operation of one or more trained models via the ML operation module 144).

In some aspects, the SVAAS 104 may include one or more trained ML models which may be stored in database 126, such as trained ML models 150A, 150B, 150C, 150D, 150E. In some aspects, each ML model may have different functionality and/or may be trained using different training data. In some aspects, one or more ML models may have at least some of the same functionality and/or be trained using at least some of the same training data.

Trained ML models 150A, 150B, 150C may in some aspects collectively be described as trained assessment ML models, trained to generate one or more assessment metrics from surgical input data which may be used by the SVAAS 104 to generate a performance assessment of a surgeon. In some aspects, each of the trained ML assessment models 150A, 150B, 150C may generate one or more metrics which the other assessment trained ML modules do not, for example: trained surgical instrument assessment ML model 150A may generate one or more surgical instrument metrics; trained surgical phase assessment ML model 150B may generate one or more surgical phase metrics; and trained anterior capsulotomy (AC) assessment ML model 150C may generate one or more AC metrics. In other aspects, each trained assessment ML model, or any trained ML model or SVAAS 104, may generate one or more of the same assessment metrics and/or outputs as another trained ML model.

In another aspect, trained semantic segmentation ML model 150D may generate a semantic segmentation subset data. In yet another aspect, trained video editing ML model 150E may generate an edited surgical video data.

In an embodiment of the present systems and methods discussed herein, the SVAAS 104 may use one or more trained ML models to analyze a video of a surgery (surgical video data), generate various assessment metrics of the surgery, surgeon, and/or aspects thereof, included in the surgical video data, and generate a performance assessment of the surgeon from the assessment metrics.

In an aspect, the SVAAS 104 may train one or more ML models using training data. For example, the SVAAS 104 may receive training data from a user device 102 over a network 110, which the SVAAS 104 may store in database 126. The training data may be video data including one or more images capturing at least a portion of surgical procedure, for example video data from an ophthalmic surgery (historical ophthalmic surgery data), such as a cataract surgery. The training data may have been processed in one or more ways, for example labeling the data/images, semantically segmenting images, and/or any other processing suitable for training data.

At least a portion of the training data may be loaded by SVAAS 104 (e.g., via processor 120) into ML model training module 142 to train one or more ML models, such as ML models for generating one or more metrics related to a surgery and/or surgeon, assessing the performance of a surgeon, editing a video, and/or semantically segmenting an image, as well as any other suitable purpose based at least upon the training data, especially as it relates to analyzing a surgeon, a surgery, and/or otherwise performance assessments. Once trained, one or more trained ML models (e.g., trained ML models 150A, 150B, 150C, 150D, 150E) may be stored in memory 122 and/or database 126, loaded by the SVAAS 104 into ML operation module 144 at runtime, and provided one or more data inputs to process, which in some examples may include surgical video data.

In an aspect, each of the trained assessment ML models 150A, 150B, 150C may generate one or more assessment metrics, which the SVAAS 104 may use to generate a performance assessment of the surgeon. In some aspects, assessment metrics generated by trained surgical instrument assessment ML model 150A may relate to one or more surgical instruments used in the surgery depicted in the surgical video data, and may include the order the instruments are used, the location of the instrument in an image and/or the duration the instrument is used in a surgery. In other aspects, assessment metrics generated by trained surgical phase assessment ML model 150B may relate to the phase of the surgery, which may include an order or a surgical step or the duration of a surgical step in one or more phases of a surgery, such as a cataract surgery. In other aspects, assessment metrics generated by trained AC assessment ML model may relate to the AC, which may include the size, centration, eccentricity, circularity, smoothness of a capsulorrhexis and/or the fluidity of a capsulorrhexis creation.

Once generated, the one or more assessment metrics may be used by the SVAAS 104 to generate a performance assessment of the surgeon. In some aspects, the performance assessment may be used for a variety of purposes beyond providing an assessment of the performance of surgeon during a surgery, such as real-time guidance of a surgery and/or providing one or more warnings related to a surgery while the surgery is occurring; assessing the trajectory of the surgeon's skill over time; providing board certification; credentialing at a medical establishment; and/or pay-for-performance by an insurance carrier, as well as other suitable purposes.

In some embodiments, the SVAAS 104 may train other ML models using ML model training module 142, such as trained semantic segmentation ML model 150D. The semantic segmentation ML model 150D may be trained using historical semantic segmentation data including one or more classified capsulorrhexis images. Once trained, the trained semantic segmentation ML model 150D may be loaded at runtime into ML operation module 144, process the surgical video data to generate segmentation subset data including semantically segmented images of a capsulorrhexis. The semantic segmentation subset data may be uses by the SVAAS 104 trained assessment ML models 150A, 150B, 150C to generate one or more assessment metrics, for example as AC metrics. In an example, using the semantic segmentation subset data as an input to the trained AC assessment ML module 150C may be beneficial due to the semantically segmented images of a capsulorrhexis which the SVAAS 104 may be more adept to score and/or assess.

In some aspects, the SVAAS 104 may use ML model training module 142 to train video editing ML model 150E. The training may use historical surgical phase activity data including one or more images of phases of a surgery, for example in the case of a cataract surgery, this may include paracentesis, medication injection, viscoelastic insertion, main wound, capsulorrhexis initiation, capsulorrhexis completion, hydrodissection, phacoemulsification, cortical removal, lens insertion, a viscoelastic removal, and wound closure. The trained video editing ML module 150E may use the surgical video data as input to generate an edited surgical video data in which phases of inactivity shown in the surgical video data have been removed. In some aspects, the edited surgical video may be provided by the SVAAS 104 to user device 102, e.g., to be used for teaching a specific phase of a surgical procedure by an instructor. In another aspect, the edited surgical video may be processed by trained assessment ML models 150A, 150B, 150C to generate assessment metrics. In an aspect, using the edited surgical video data may beneficially shorten processing time and reduce processing requirements of the SVAAS 104.

While ophthalmic surgery, and more specifically at times a cataract surgery and an AC procedure, have been discussed as example surgeries, the SVAAS 104, systems, methods and techniques disclosed may be used for other types of surgeries. Similarly, the SVAAS 104 may be used for assessing performance of any type of surgeon and/or any performance assessment beyond the surgical/medical profession.

### Exemplary High-Level System Flow

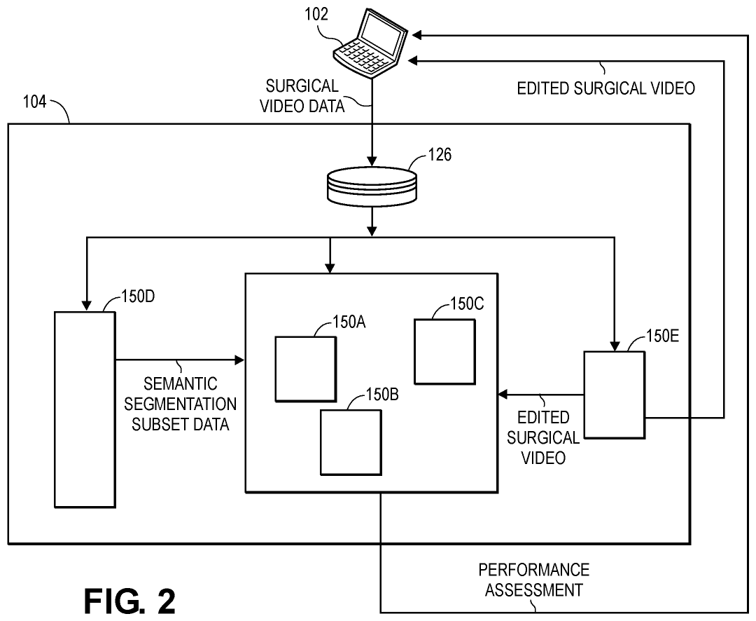

FIG. 2 is an exemplary block diagram depicting a high-level system flow using ML to analyze a surgical video to assess performance of a surgeon conducting a surgical procedure. In general, the system flow may be carried out by the components of the computing environment 100.

According to an embodiment, the SVAAS 104 may receive surgical video data including one or more images capturing at least a portion of an ophthalmic surgical procedure from a user device 102. The SVAAS 104 may store the surgical video data, e.g., in memory 122 and/or database 126.

In an aspect, the user may provide the surgical video data to the SVAAS 104 over network 110 via user device 102. In an example, the user device 102 may include a desktop computer, a laptop, a smartphone, a wearable device such as a smartwatch, augmented/virtual/mixed reality head-mounted display, etc., operably connected to the SVAAS 104, e.g., over a network 110.

In one example, the user may interact with the SVAAS 104 via a web browser, software application, and/or operating system hosted/run either locally on the user device 102, and/or remotely (e.g., in the cloud and/or served from the SVAAS 104). This may include a graphical user interface (GUI) or other user interface to provide unidirectional or bidirectional communication, input, and/or transfer of information and/or commands between the user device 102 and the SVAAS 104.

In other aspects, the user device 102 may comprise a user interface of the SVAAS 104, e.g., a GUI presented by and/or on the SVAAS 104 using a display or touchscreen. Further to this example, the user device 102 may allow a user to provide surgical video data to the SVAAS 104 without the use of a separate computing device. For example, using a GUI on the SVAAS 104, a user may provide a surgical video data file via the internet or the cloud. In another aspect, the user may use the GUI of the SVAAS 104 to access a storage device which may interface with the SVAAS 104 via I/O module 146 to provide the surgical video data, e.g., a CD-ROM, DVD-ROM, USB drive, flash drive, hard disk drive (HDD), and/or the like.

In some aspects, when providing the surgical video data to the SVAAS 104, or at any other time, the user may be required, or have a choice, to select one or more options associated with the operation of the SVAAS 104. In an aspect, the options may be presented to the user via a GUI on user device 102, as well as any other means of interacting with the SVAAS 104, as discussed herein.

In some aspects, the one or more options the SVAAS 104 may present to a user may be selection of: metrics to generate; performance assessments to generate; whether a surgical video data should be edited, and if so, what editing criteria; surgical phases to assess, whether a surgical video data should be semantically segmented; board certifications; credentialing options; pay-per-performance options; recipients of data, information, metrics and/or assessments generated; as well as any other suitable options associated with one or more operational parameters of the SVAAS 104. In other aspects, the SVAAS 104 may not require user selections from a user and may operate autonomously, and in others the SVAAS 104 may require some user input to operate semi-autonomously.

The SVAAS 104 may process the surgical video data using one or more trained assessment ML models, e.g., 150A, 150B, 150C, to generate one or more assessment metrics. In an aspect, the one or more trained assessment ML models 150A, 150B, 150C may be trained by ML model training module 142 using historical ophthalmic surgery data, which may be stored in memory 122 and/or database 126.

The historical ophthalmic surgery data which may be used as training data for the one or more assessment ML models 150A, 150B, 150C, as well as any other ML model of the SVAAS 104, and may include one or more images indicating (e.g., using labels) one or more of an instrument presence, an instrument identification, an instrument color, an instrument material, a surgical step identification, a surgical phase identification, a capsulorrhexis identification, a limbus identification, a pupil identification, a purkinje image identification, an anatomical landmark identification, or an anatomical change identification.

Once trained, the one or more trained assessment ML models 150A, 150B, 150C may be loaded at runtime into ML operation module 144, process the user's surgical video data which may be stored in database 126, and generate one or more assessment metrics which may include one or more of a surgical instrument metric, a surgical phase metric, and/or an AC metric. The SVAAS 104 may intelligently and/or autonomously select which of the one or more trained assessment ML models 150A, 150B, 150C to process the surgical video data; may make selections semi-autonomously, e.g., based in part upon a user input which may be received at any time; and/or may make selections only based upon one or more inputs which may be received at any time, e.g., based upon the metrics and/or performance assessments a user may select when providing the surgical video data to the SVAAS 104.

In an aspect, surgical instrument assessment ML model 150A may generate one or more surgical instrument metrics. The surgical instrument ML model 150A may be trained using the historical ophthalmic surgery data in some aspects, and/or in other aspects only a portion or a subset of the historical ophthalmic surgery data which may be pertinent to a surgical instrument e.g., instrument subset data, either or both of which may be store in memory 122 and/or database 126. The instrument subset data may include one or more images indicating (e.g., via labels) one or more of: the presence of a surgical instrument in an image (instrument presence); the identification of the surgical instrument in an image (instrument identification); the color of the surgical instrument (instrument color); and/or the material the instrument is comprised of (instrument material), as well as any other suitable information which may assist an ML model in generating a surgical instrument metric. In one example, CAD data of a surgical instrument may be used as instrument subset data which include 3D data providing rendering and pose of an instrument.

In an aspect, example surgical instrument metrics the trained surgical instrument assessment ML model 150A may generate may include identifying one or more instruments and the order in which each surgical instrument is used during a surgical procedure (instrument ordering); the location of one or more surgical instruments (instrument location) which may include identification of one or more instruments and the instrument's location with respect to another identified instrument, an identified landmark, the image frame, or any other suitable manner of identifying instrument location/localization, to name but a few; and/or the duration an identified instrument is used during a phase and/or step in a procedure (instrument duration). Instruments which the SVAAS 104 may be able to detect based on training may include one or more of a cystotome, a chopper/second instrument, an irrigation/aspiration handpiece, a keratome, a lens injector, a paracentesis blade, a phacoemulsification handpiece, utrata forceps, a stabilizer, a lens dialer, and/or a cannula, however, other surgical instruments may also be trained and detected by an appropriate ML model, including those unrelated to ophthalmic surgery.

In another aspect trained surgical phase assessment ML model 150B may generate one or more surgical phase metrics. The surgical phase assessment ML model 150B may be trained using the historical ophthalmic surgery data in some aspects, and/or in other aspects only a portion or a subset data of the historical ophthalmic surgery data which may be pertinent to a surgical phase, e.g., a surgical phase subset data. Surgical phase subset data may include one or more images indicating (e.g., labeled) one or more of: the step in a surgical procedure (surgical step identification); the phase of a surgical procedure (surgical phase identification); as well as any other suitable indications which may assist an ML model in generating a surgical phase metric.

In an example related to cataract surgery, surgical phases may include paracentesis, medication injection, viscoelastic insertion, main wound, capsulorrhexis initiation, capsulorrhexis completion, hydrodissection, phacoemulsification, cortical removal, lens insertion, viscoelastic removal, and/or a wound closure. In an aspect, one or more steps may comprise one or more phases of surgery. Surgical phase metrics the SVAAS 104 may generate using the trained surgical instrument assessment ML model 150B may include the order in which certain surgical steps occur and/or are completed (surgical step order) and/or the duration of time it takes to complete one or more surgical steps (surgical step duration).

In another aspect, trained AC assessment ML model 150C may generate one or more AC metrics. The AC assessment ML model 150C may be trained using the historical ophthalmic surgery data in some aspects, and/or in other aspects only a portion or a subset of the historical ophthalmic surgery data which may be pertinent to an AC, e.g., a capsulotomy subset data which may include one or more images indicating (e.g., with labels) one or more of: identifying the capsulorrhexis (capsulorrhexis identification); identifying the pupil (pupil identification); identifying the limbus location (limbus location); identifying the purkinje image location (purkinje image location); identifying an anatomical landmark (anatomical landmark); and/or identifying an anatomical change (anatomical change), e.g., an anatomical change in the capsulorrhexis, as well as any other suitable indications which may assist an ML model in generating an AC metric. AC metrics the SVAAS 104 may generate using the trained AC assessment ML model 150C may include a capsulorrhexis size, a capsulorrhexis centration, a capsulorrhexis eccentricity, a capsulorrhexis circularity, a capsulorrhexis smoothness, or a fluidity of a capsulorrhexis creation. One or more of the AC metrics may be used by the SVAAS 104 to assess the performance of the surgeon.

The SVAAS 104 may generate based upon at least the one or more assessment metrics, a performance assessment. In some aspects, one or more of the surgical instrument metrics may be used by the SVAAS 104 to assess the performance of the surgeon. For example, if a surgeon lacks stability or fluidity of movement of a surgical instrument used to conduct the AC, this metric may indicate the surgeon lacks experience and/or expertise, resulting in a performance assessment indicative of inexperience, or any other suitable feedback.

In some aspects, one or more of the surgical phase metrics may be used by the SVAAS 104 to assess the performance of the surgeon. For example, if a surgeon takes steps to conduct a wound closure before the procedure involving the wound is complete, this metric may indicate the surgeon lacks experience and/or expertise. With respect to surgical step duration, if a surgeon completes all steps of a surgery phase in the correct order and in an amount of time which may evidence efficiency, the SVAAS 104 may generate a performance assessment indicative of expertise, or any other suitable feedback.

In some aspects, one or more of the AC metrics may be used by the SVAAS 104 to assess the performance of the surgeon. For example, if a surgeon creates a capsulorrhexis which is centered and smooth, these metrics may indicate the surgeon possess experience and/or expertise and the SVAAS 104 may generate a performance assessment indicative of same. In another example, if the capsulorrhexis is off-centered and unusually small, the SVAAS 104 may provide an assessment that more training is needed for the surgeon, or any other suitable feedback.

In an aspect, the SVAAS 104 may use a four-category grading rubric for capsulorrhexis creation, grading the four components between 1 and 10 and defined as follows: centration—the extent to which the capsulorrhexis is centered with regard to the intraoperative dilated pupil; size—the closeness to which the size of the capsulorrhexis is ideal for the size of the optic (approximately 5 mm in this dataset); circularity—the extent to which the circumference of the capsulorrhexis follows the path of a circle; and eccentricity—the extent to which the major and minor axes of completed capsulorrhexis matched.

The SVAAS 104 may generate a performance assessment of the surgeon and provide to it user device 102. In some aspects, the performance assessment may be stored in memory 122 and/or database 126. In some aspects, the performance assessment of the surgeon is based upon at least the one or more assessment metrics. In some examples, the performance assessment may include one or more of the following assessments: a skill level assessment, e.g., providing a score ranking the surgeon as a novice, beginner, advanced beginner, or competent, which may be similar to the ICO-Ophthalmology Surgical Competency Assessment Rubric-Phacoemulsification; a phase of surgery duration assessment which may provide feedback on how long a step or phase of surgery generally takes, e.g., a wound closure for a cataract surgery generally takes 60-120 seconds for various levels of tenure for a surgeon; a surgical quality assessment, e.g., one which considers multiple metrics when making an overall assessment of the surgical quality such as capsulorrhexis size, diameter, circumference, circularity, centeredness, smoothness and/or how clean an aspect of a surgical cut, incision, excision, or the like may be; a skill progression assessment which may track the trajectory of one or more skills of a surgeon over time; an AC assessment which in an aspect may consider one or more metrics associated with conducting an AC; a board certification assessment which may consider one or more criteria needed in attaining a board certification such that the SVAAS 104 may be able to, or suggest, board certification of a surgeon; a credentialing assessment e.g., which may allow for credentialing of a surgeon at a place of work such as a hospital; a pay-for-performance assessment for example which may provide insurance carriers data related to the performance of one or more surgeons; and/or an early warning assessment which, in an aspect, may detect if the early warning assessment reaches a threshold which requires the SVAAS 104 to provide a warning in real-time during a surgical procedure to mitigate harm, for example providing an visual or auditory alarm on a user device 102 that a step of a procedure has been performed so inadequately that the patient may be in danger if not immediately remedied. In other aspects, the performance assessment may include other criteria, and/or exclude the above-noted criteria, and include other performance assessments which would be suitable for assessing the skill of a surgeon.

In some aspects as noted above, the one or more performance assessments may include qualitative or quantitative feedback. If using a score, one or more assessment may be scored individually, averaged, or calculated in any suitable manner. Assessments may be made based upon the relative skill of the surgeon anywhere between trainee to expert. For example, a board certification assessment may only be provided once a surgeon has attained at least a threshold level of skill. Any form of assessment may be provided according to the systems, methods, and techniques of the present inventions.

The performance assessment may be provided asynchronously and/or at various times during and/or after a surgery, and/or receiving surgical video data. As referenced above, some assessments may be time-sensitive and worthy of immediate feedback, e.g., to a user device 102 by the SVAAS 104, such as warning of imminent danger during a surgery or as a real-time surgical guidance system.

In another aspect, an assessment may be provided within a reasonable time frame soon after the surgery, for example feedback on a recently conducted surgery which the surgeon may be able to receive and consider before another upcoming similar surgery. In another aspect, an assessment may be used for teaching and/or academic purposes which may require the SVAAS 104 to provide feedback and assessment in the near term.

In yet another aspect, an assessment may be provided over a longer term, such as for a board certification which may need to consider surgeries over a substantially longer period of time. In another instance, the performance assessment may be used in an evaluation of the impact of surgical quality on patient outcomes, linking pre-operative data and postoperative data to make such an evaluation.

In an embodiment, the SVAAS 104 may process the surgical video data using a trained semantic segmentation ML model 150D to generate a semantic segmentation subset data including one or more images indicating a semantic segmentation of a capsulorrhexis. The semantic segmentation ML model 150D may be trained using historical semantic segmentation data including at least one or more images of a classified capsulorrhexis. Classification may include identification, ground truth annotation, outlining and/or bound boxing one or more images and/or frames of anatomy or landmarks associated with a capsulorrhexis, e.g., a capsulorrhexis, the limbus, the purkinje image, as well as any other suitable anatomy or landmarks.

The SVAAS 104 may process the semantic segmentation subset data and/or the surgical video data using one or more trained assessment ML models (e.g., the AC assessment ML model 150C) to generate one or more assessment metrics.

In an embodiment, SVAAS 104 may process the surgical video data using a trained video editing ML model 150E to generate an edited surgical video data. The trained video editing ML model 150E may be trained using historical surgical phase activity data. In an aspect, the historical surgical phase activity data may include one or more images indicating one or more surgical phases which may include paracentesis, a medication injection, a viscoelastic insertion, a main wound, a capsulorrhexis initiation, a capsulorrhexis completion, a hydrodissection, a phacoemulsification, a cortical removal, a lens insertion, a viscoelastic removal, or a wound closure.

When processing the surgical video data by trained video editing ML model 150E, one or more images capturing phase inactivity may be removed, generating an edited surgical video data. In some aspects, phase inactivity may include images of phases of surgery which may not affect the ability to the SVAAS 104 to generate accurate assessment metrics and/or performance assessments, and thus the removal of such phases of inactivity may be beneficial to the SVAAS 104 as compared the unedited surgical video data in reducing processing time and storage requirements.

In some embodiments, once video editing ML model 150E is trained, it may be able to remove one or more images capturing phase inactivity by comparing temporal aspects of the surgical video data such as frames before and after surgical activity, and spatial aspects of the surgical video data, such as localization of instruments and/or identification of instruments, steps and/or phases of surgery, as well as any other suitable aspects of the surgical video data.

In some embodiments, a user may only want the edited video data to contain one specific phase of surgery, e.g., a capsulorrhexis completion of a cataract surgery. For example, an instructor may be teaching a class on this particular phase of surgery and may require multiple edited videos to present to the class showing various surgeons actually conducting only that procedure. In such an example, the user may provide to the SVAAS 104, e.g., via user device 102, an input selection that trained video editing ML model 150E should generate an edited surgical data consisting only of a capsulorrhexis completion phase of a cataract surgery.

In an aspect, once the edited surgical video data is generated by trained video editing ML model 150E, in an aspect it is provided to the user device, e.g., the edited video data to be used for teaching purposes. In one aspect, the edited surgical video may be processed by one or more trained ML models of the SVAAS 104, e.g., to generate one or more assessment metrics.

Although in some embodiments one or more trained ML models are contemplated having specific functionality (e.g., generating assessment metrics, editing surgical video data, semantically segmenting images), in other embodiments systems and methods may include fewer trained ML models, or single trained ML model, which may have the same functionality. In an example, a single trained ML model may generate the same outputs as trained semantic segmentation ML model and trained AC assessment ML model combined. Various combinations of trained ML models and associated functionality are contemplated as within the scope of the SVAAS 104.

In some embodiments, a model or trained model may be comprised of one or more models, algorithms, architectures and/or layers, for example residual neural networks, recurrent neural networks, convolutional neural networks and models such Densenet169, an inflated 3D model, and/or YOLOv4, to name but a few.

Additionally, although historical surgical phase activity data may be discussed in the context of cataract or ophthalmic surgery, any type of surgery may be contemplated by the present systems, methods and techniques.

### Exemplary Training Database

When training one or more ML models and/or algorithms, voluminous, high-quality data may be beneficial. This high-quality data may include labeled ground truth training data, whereby training data may also include testing and/or validation data. Additionally, creating high-quality labels for data may enable an ML system to provide both low-level and high-level recognition tasks using the same data. In an example, annotations of surgical video data at full frame rate (e.g., 30 fps) and high-definition resolution (e.g., 1980×1020 pixels) may allow for development of ML systems which provide (i) offline processing which may be used teaching and/or feedback of surgical procedures after they have been completed. For example offline processing may allow predictions about a surgical procedure which may include looking at voluminous data in the future and in the past with respect to a prediction; (ii) online processing which may have moderate latency for results from an ML system requiring only to process temporal data which may include only frames immediately after a frame of interest; and (iii) real-time processing which may be beneficial for low latency ML results, such as surgical guidance which may only require processing a previous frame of interest.

In an aspect, training data for the various ML models discussed herein may be created by surgical imaging devices such as Zeiss high-definition resolution 1-chip imaging sensors integrated into ceiling-mounted Zeiss Lumera 700 operating microscopes, or as another example Karl Storz AIDA recording devices to obtain high-definition resolution surgical video recordings. In an embodiment, a surgical imaging database (which may be used as, for, or be processed to become, a training database) may contain over 4 million frames of surgical video data.

In the case of ML related to surgical instruments, such as trained surgical instrument ML model 150A which may generate instrument identification, instruments presence, and instrument material to name but a few examples, it may be beneficial to augment and/or transform relevant training data, images, and/or video in various ways to improve the performance of an ML model. This may include, but is not limited to, image resizing, rotating, shifting, shearing, zooming, horizontal/vertical flipping, and/or rescaling.

With respect to ML models related to an AC and identifying phases and/or steps of a surgical procedure, identifying one or more images and/or frames in training data which may provide a view of, as an example, a capsulorrhexis just after its completion, but prior to the hydrodissection phase of surgery, may be beneficial. Continuing with this example, having training data with adequate visualization and labeling of the limbus, pupil, and capsulorrhexis without surgical instruments obscuring the view may also be beneficial.

Classification of training images may also be beneficial to the operation of an ML model. In one aspect, this may include having all pixels of, on, and within a traced contour classified as belonging to a capsulorrhexis, while all other pixels within the image may be classified as not belonging to the capsulorrhexis. This may be beneficial when creating ground truth masks for an ML model processing data which may relate to a capsulorrhexis or AC, such as for historical semantic segmentation data which may be used to train semantic segmentation ML model 150D.

In another aspect, it may be beneficial for the training data to have high-quality images which have adequate lighting and resolution, e.g., images which do not contain reflections, are sufficiently bright and/or of a resolution to detect and/or distinguish what is being shown in the image. Additionally, it may be beneficial for a training image to have an appropriate field of view which captures all objects and/or aspects which may be considered beneficial to the training data, e.g., an image which shows the entire instrument during a surgical procedure rather than having it partially out of frame.

Additionally, recording, tagging, labeling, and/or annotating one or more relevant characteristics of an image in a database, such as database 126 which may contain ML model training data, may be beneficial, e.g., duration of a phase of surgery, instrument presence, surgical step identification, limbus location, purkinje image location.

Including data from one or more databases of the same surgical procedure may also be beneficial in determining one or more metrics and/or assessing a surgeon and/or surgery. For example, associating relevant preoperative, perioperative and/or postoperative data, may allow an ML model to evaluate the longer-term outcome of a surgery whereas evaluating data obtained only during the surgery may not. Examples of preoperative, perioperative and/or postoperative data may include exam findings, clinical notes, biometry measures, corneal topography, preoperative and postoperative refractions, visual acuity, intraocular pressures, as well as any other data which may be suitable to assess a surgery and/or surgeon.

While ophthalmic, cataract, capsulorrhexis and/or AC data, surgeries, and surgical instruments have been discussed herein, the above techniques may be applicable to data used for other types of surgeries, medical procedures, metrics, assessments and the like.

### Exemplary Identification of Surgical Instruments

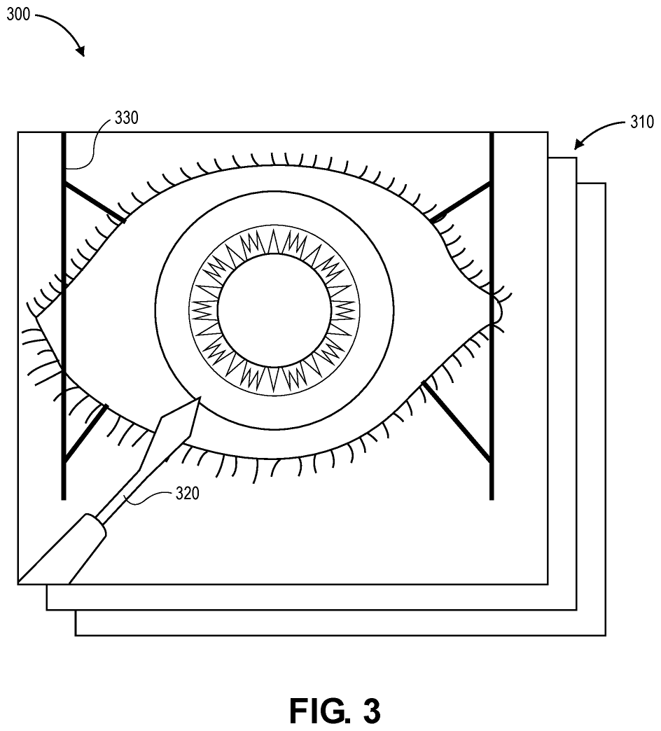

FIG. 3 depicts an exemplary image 300 in which machine learning (ML) identifies one or more surgical instruments in data from a surgical video.

In an embodiment according to FIG. 3, SVAAS 104 may train a surgical instrument assessment ML model via ML model training module 142 using historical ophthalmic surgery data and/or surgical instrument subset data, which may be stored in database 126. Once trained, SVAAS 104 may store trained surgical instrument assessment ML model 150A in database 126.

In one aspect, ML surgical instrument identification may be possible using one or more lightweight convolutional neural networks (CNNs) trained using large high-quality datasets such as the training data discussed supra, to achieve state-of-the-art performance without a complex model architecture, e.g., using an ML comprising a dense neural network (NN) layer ensembled with a YOLOv4 model for object detection.

In an additional aspect, different approaches may be used depending on the number of instruments detected in an image, e.g., detecting one instrument may include a bounding box which has the highest combined confidence score and classification score for detection, and labeling using the dense NN as opposed to using the ensemble model mentioned above which may be used for detecting multiple images.

Continuing with the example of FIG. 3, the historical ophthalmic surgery data and/or surgical instrument subset data may include one or more images indicating (e.g., via labels) one or more of: the presence of a surgical instrument in an image (instrument presence); the identification of the surgical instrument in an image (instrument identification); the color of the surgical instrument (instrument color); and/or the material the instrument is comprised of (instrument material), as well as any other suitable information which may assist an ML model in generating a surgical instrument metric.

Being that the same surgical instrument may have very different appearances based upon considerations such as manufacturer, material, size, to name but a few, e.g., a handpiece with polymer tip vs. silicone tip, the ability of a trained ML model (e.g., surgical instrument assessment ML model 150A) to generalize and/or have the ability to adapt properly to new, previously unseen data drawn from the same distribution as the one used to create the model, may be affected by the data used for training the ML model.

In an aspect of SVAAS 104, the historical ophthalmic surgery data and/or surgical instrument subset data may include multiple examples of potential representations of a given surgical instrument type, which may increase the accuracy of an ML model, such as surgical instrument assessment ML model 150A, to detect the given surgical instrument. As an example, if using ML to analyze a surgical video and assess performance of a surgeon, it may be beneficial to train the model using data containing images of surgical instruments from multiple manufacturers with a variety configurations, in various poses, which in an example of a cataract surgery may include instruments such as a cystotome, a chopper/second instrument, an irrigation/aspiration handpiece, a keratome, a lens injector, a paracentesis blade, a phacoemulsification handpiece, utrata forceps, a stabilizer, a lens dialer, and/or a cannula. However, other surgeries and surgical instruments may also be trained and detected by an appropriate ML model, including those unrelated to ophthalmic surgery.

Continuing with the example of FIG. 3, at runtime SVAAS 104 may load trained surgical instrument assessment ML model 150A from database 126 into ML operation module 144 to process surgical video data 310 composed of multiple images. Trained surgical instrument assessment ML model 150A may generate one or more surgical instrument metrics when evaluating the multiple images of surgical video data 310, which may include ordering based upon identifying the keratome 320 and stabilizer 330, as well as the order in which each appears throughout the surgical video data 310. This may also include instrument duration based upon the duration of time the keratome 320 and stabilizer 330 appeared throughout the video depicted by surgical video data 310.

In an aspect, the accurate identification of one or more surgical instruments, and the metrics generated by trained surgical instrument assessment ML model 150A, may allow the SVAAS 104 to generate a performance assessment for a surgeon performing the surgical procedure depicted in surgical video data 310. This may include, e.g., metrics indicating the ordering, duration, and location of the surgical instruments 320 and 330, and the expertise of the surgeon performing the procedure depicted in surgical video data 310. In another aspect, SVAAS 104 may provide a performance assessment in real-time using instrument identification and predict that a complication may arise during the procedure and immediately provide a warning to the surgeon, e.g., via user device 102.

### Exemplary Surgical Video Editing

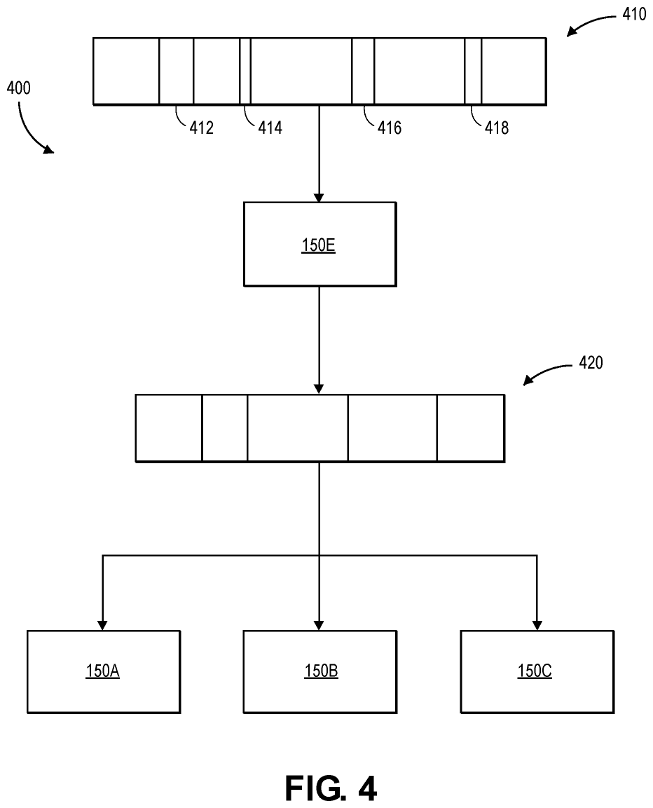

FIG. 4 depicts an exemplary block diagram 400 in which the SVAAS 104 may use ML, e.g., via trained video editing ML model 150E, to generate edited surgical video data 420 by identifying and removing images of phase inactivity 412, 414, 416, 418 in surgical video data 310.

In an aspect, the trained video editing ML model 150E may include an ensemble model consisting of a trained Densenet169 convolutional NN and an inflated 3D (I3D) model trained by combining the second to last layer output of each model as the input to two fully connected layers of size 128 and 32 nodes.

In an embodiment, the SVAAS 104 may receive from a user device a surgical video data 310 comprising of a stream of images 410 of a surgery. A trained video editing ML model 150E, which may be trained using historical surgical phase activity data, may be stored in database 126 and/or may be generated by ML model training module 142. At runtime when the SVAAS 104 receives the stream of images 410, trained video editing ML model 150E may be loaded into ML operation module 144 and process the surgical video data 310; identify images of phase inactivity 412, 414, 416, 418 from steam of images 410; remove images of phase inactivity 412, 414, 416, 418; and generate an edited surgical video data 420 which may be provided to a user device (e.g., user device 102) in some aspects, and/or processed by one or more trained ML models, such as trained assessment ML models 150A, 150B, 150C, in other aspects.

In an embodiment, trained video editing ML model 150E may be able to identify based upon the historical surgical phase activity data, twelve distinct phases of a cataract surgery, which may include a paracentesis, a medication injection, a viscoelastic insertion, a main wound, a capsulorrhexis initiation, a capsulorrhexis completion, a hydrodissection, a phacoemulsification, a cortical removal, a lens insertion, a viscoelastic removal, and a wound closure. However, surgical steps and or phases or other surgeries and/or medical procedures may likewise be detected by an appropriately trained ML model. In an aspect, video data which is not considered surgical phase activity data may be considered phase inactivity,

In some aspects, the trained video editing ML model 150E may identify individual images/frames as phase inactivity using spatial and/or temporal characteristics of each image/frame. One example of phase inactivity may include select images of a surgeon handing one instrument off to an assistant and then being handed another instrument such that no crucial surgical steps are shown in the select images.

In another aspect, phase inactivity may include one or more images which are irrelevant to a subsequent ML model which may use the edited surgical data as an input, for example an ML model which assesses qualities of a capsulorrhexis completion phase of a cataract surgery may not have use for images showing the final wound closing phase of the surgery, considering them to be phase inactivity.

In certain aspects inputting edited surgical video data 420 into another and/or subsequent trained ML models may provide better performance by the other/subsequent ML models and/or SVAAS 104. For example, processing a smaller edited video data 420 may take less time and computational resources for the SVAAS 104 than a larger, unedited video, e.g., a surgical video data 310 comprising of a stream of images 410.

In another aspect, the edited surgical video data 420 may be used for teaching purposes where an instructor only wants a video of two specific steps of a ten-step surgical procedure. The parameters of such a request may be provided to the SVAAS 104 via user device 102, and the trained video editing ML model 150E may edit the surgical video data 310 stream of images 410 accordingly, treating all images which do not show the requisite two steps as phase inactivity.

In one embodiment according to FIG. 4, the trained video editing ML model 150E may identify one or more phases of a surgery, edit a surgical video data stream 310 to generate an edited surgical video data 420 removing phase inactivity. The edited surgical video data 420 may be used as input to one or more trained assessment ML models 150A, 150B, 150C to generate one or more assessment metrics which may analyze qualitative and quantitative aspects of a surgical phase. One metric that may be generated using the edited surgical video data 420 is the time spent in a surgical phase and/or surgical step, as well as the order of surgical steps performed by the surgeon.

### Exemplary Capsulorrhexis Semantic Segmentation

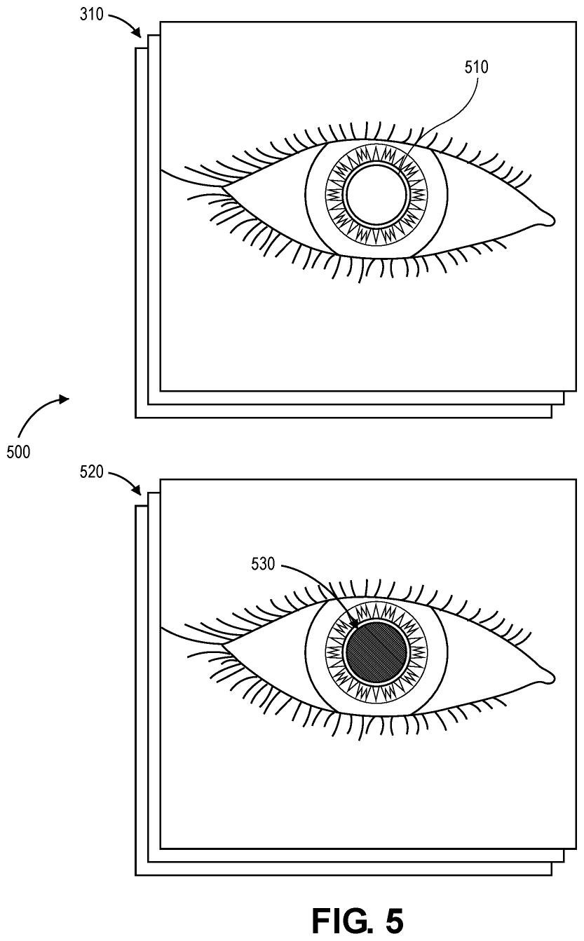

FIG. 5 depicts an exemplary block diagram 500 in which the SVAAS 104 may use ML, e.g., via trained semantic segmentation ML model 150D, to process a surgical video data 310 displaying a capsulorrhexis 510, and generate semantic subset data 520 which may include one or more images of a capsulorrhexis 510 which are semantically segmented 530 and/or classified.

The quality of a continuous curvilinear capsulorrhexis (CCC) depends at least in part upon the morphological characteristics of the capsulorrhexis 510, and accordingly, analysis of a surgical video to identify characteristics of a completed capsulorrhexis 510 may be an important step in assessing the performance of a surgeon.

In an aspect according to FIG. 5, the SVAAS 104 may process a surgical video data 310 displaying a capsulorrhexis 510 using a trained semantic segmentation ML model 150D to generate a semantic segmentation subset data 520 including one or more images indicating a semantic segmentation 530 of a capsulorrhexis 510. The trained semantic segmentation ML model 150D may be trained using historical semantic segmentation data including at least one or more images of a classified capsulorrhexis 510.

In an embodiment, the architecture of the trained semantic segmentation ML model 150E may include a DeepLabv3+ model having an input image which is first passed into a backbone deep convolutional neural network (e.g., utilizing ResNet50). Low level features derived from the deep CNN may be passed into a decoder module and concurrently the output from the deep CNN may be passed into an Atrous Spatial Pyramid Pooling (ASPP) module which may capture spatial features at different scales by utilizing different stride rates in parallel. The output of the ASPP module may then be up-sampled and passed into the decoder, where it is concatenated with the low level features at corresponding spatial resolution from the backbone deep CNN. The results may then be passed to 3×3 convolution layers, up-sampled by 4, and may become the output of the DeepLabv3+ network.

In an aspect, semantic segmentation subset data 520 may then be processed by one or more trained assessment ML models 150A, 150B, 150C, such as trained AC assessment ML model 150C, to generate one or more assessment metrics, such as AC metrics. The AC metrics may include morphological characteristics, which may include capsulorrhexis size, capsulorrhexis centration, capsulorrhexis eccentricity, capsulorrhexis circularity, capsulorrhexis smoothness, and/or a fluidity of a rhexis formation. In an embodiment, the SVAAS 104 may use the one or more assessment metrics and/or AC metrics as part of an objective analysis and/or assessment of cataract surgery performance, which may include, but is not limited to, longitudinal feedback (i.e., feedback on surgical performance trajectory over time).

### Exemplary Performance Assessment

In an embodiment, the SVAAS 104 may provide a video-based performance assessment of surgical skill using objective metrics related to surgical actions and characteristics. This may include temporal metrics such as time spent on certain aspects of the surgery, spatial metrics such as order and location of surgical instruments, and longitudinal analysis tracking the trajectory of surgical performance, as well as other metrics, actions and characteristics suitable for surgical skill assessment.

Specifically with respect to cataract surgery, the skillful performance of the CCC allows for stability of the capsular bag during nucleus disassembly and cortical removal. The continuity and appropriate sizing of the capsulorrhexis 510 are essential to achieving the appropriate positioning of the intraocular lens implant at the end of a cataract surgery. A capsulorrhexis 510 that is too small can result in anterior capsular phimosis and visual field constriction, while a capsulorrhexis 510 that is too large can lead to instability and tilting of the intraocular lens implant. The achievement of a smooth, continuous, round, appropriately sized capsulorrhexis 510 may be challenging for surgeons, especially while in training. The ability of the SVAAS 104 to use surgical video data 310 to identify and generate metrics related to various morphological features of a capsulorrhexis 510, as well as generate objective surgical feedback on CCC quality, may be one of many valuable features of the SVAAS 104 in assessing surgical performance for cataract surgery.

In an embodiment, SVAAS 104 receives surgical video data 310 from a user device 102 to process and analyze using one or more trained ML models, 150A, 150B, 150C, 150D, 150E. Specific ML models, e.g., trained assessment ML models 150A, 150B, 150C, may be trained to provide one or more assessment metrics based upon the surgical video data 310, the assessment metrics being used to generate a performance assessment by the SVAAS 104 to provide to user device 102. The trained assessment ML models 150A, 150B, 150C may additionally and/or alternatively process other data to generate assessment metrics, for example edited video data generated by trained video editing ML model 150E and/or semantic segmentation subset data 520 generated by trained semantic segmentation ML model 150D.

The one or more assessment metrics may relate to qualitative and quantitative aspects of a surgical video via associated surgical video data 310 having one or more images capturing at least a portion of a surgical procedure, such as an ophthalmic surgical procedure and more specifically a cataract surgery, although any surgery and/or surgical video data 310 may be received by SVAAS 104.

In some embodiments, trained assessment ML models 150A, 150B, 150C, may be trained to provide different assessment metrics. In one example, trained surgical instrument assessment ML model 150A may be trained to generate surgical instrument metrics which may include one or more of instrument ordering, instrument location, and/or instrument duration. In another example, trained surgical phase assessment ML model 150B may be trained to generate surgical phase metrics which may include one or more of a surgical step order and/or surgical step duration. In yet another example, trained AC assessment ML model 150C may be trained to generate AC metrics which may include one or more of a capsulorrhexis size, a capsulorrhexis centration, a capsulorrhexis eccentricity, a capsulorrhexis circularity, a capsulorrhexis smoothness, and/or a fluidity of a rhexis formation. However, one or more of the trained ML models of the SVAAS 104 may each and/or collectively provide the same, similar, or overlapping assessment metrics.

Using the assessment metrics, the SVAAS 104 may provide a performance assessment of a surgeon to a user device 102. User device 102 may be any of a number of devices, e.g., a surgeon's smartphone; a computer credentialing system at a hospital; a grading system at a medical school; a computer system in a classroom; a surgical system receiving a warning in real-time about a surgery the surgeon is performing; and/or a board certification computer system, as well as any other suitable user devices.

The performance assessment may include one or more of a skill level assessment, a phase of surgery duration assessment, a surgical quality assessment, a skill progression assessment, an AC assessment, a board certification assessment, a credentialing assessment, a pay-for-performance assessment, or an early warning assessment.

The performance assessments may include: scoring; rating; ranking; qualitative assessment(s)/aspect(s); quantitative assessment(s)/aspect(s); cumulative assessment(s)/aspect(s); verbal, written, textual, alphabetical, numeric, alphanumeric, electronic, graphical, pictorial, and/or multimedia assessments; as well as any other suitable assessment.

In some aspects, each assessment metric generated may itself be considered a performance assessment which may be provided to a user device. In some aspects, one or more metrics may be considered a performance assessment which may be provided to a user device as either a single performance assessment and/or multiple performance assessments. Separate performance assessments may be generated by each of the trained assessment ML models 150A, 150B, 150C, each of which may provide one or more assessments to a user, e.g., via user device 102. In some aspects, performance assessments from one or more of the trained assessment ML models 150A, 150B, 150C may be further processed by any one of the trained assessment ML models 150A, 150B, 150C and/or the SVAAS 104, and once processed, one or more assessments may be provided to a user, e.g., a surgeon via user device 102.

In another aspect, any combination and/or permutation of providing one or more performance assessments by one or more of the trained ML models 150A, 150B, 150C, 150D, 150E and/or SVAAS 104 based upon one or more assessment metrics may be contemplated by the systems, methods, and techniques disclosed.

Further, while the systems, methods and techniques disclosed generally discuss surgical performance assessment based upon surgical videos/surgical video data 310, any surgery may be contemplated by the systems, methods, and techniques disclosed, which may or may not include any type of surgery or surgeon, and/or may or may not include ophthalmic and or cataract surgery. Additionally, any type of performance assessment, including those unrelated to a surgeon and/or surgery may be contemplated by the systems, methods, and techniques disclosed.

Exemplary Method for using ML to Analyze a Surgical Video and Assess Performance

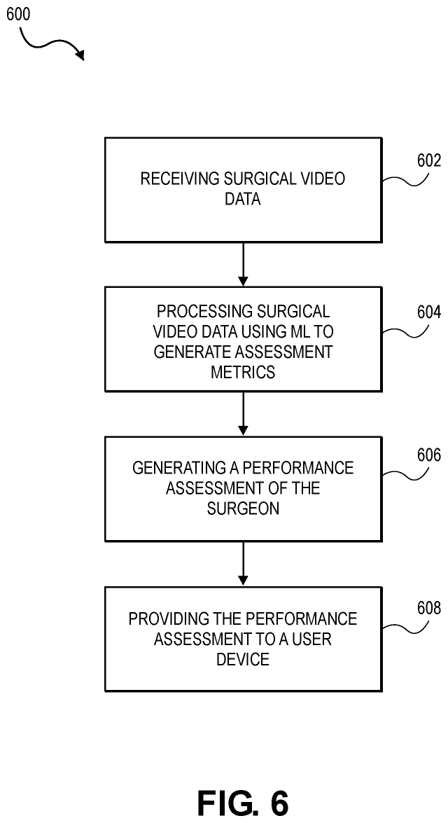

FIG. 6 is an exemplary block flow diagram depicting a computer-implemented method 600 using ML to analyze a surgical video to assess performance of a surgeon conducting a surgical procedure. In general, the method 600 may be carried out by the components of the computing environment 100.

According to an embodiment, the method 600 at block 602 may include receiving, by one or more processors 120, surgical video data 310 including one or more images capturing at least a portion of an ophthalmic surgical procedure. The SVAAS 104 may store the surgical video data 310, e.g., in memory 122 and/or database 126.

In an aspect, the user may provide the surgical video data 310 to the SVAAS 104 over a network 110 via a user device 102. In an example, the user device 102 may be operably connected to the SVAAS 104, e.g., over a network 110. In some examples, the user may interact with the SVAAS 104 via a web browser, software application, and/or operating system run either locally on the user device 102, and/or remotely (e.g., in the cloud and/or served from SVAAS 104), which may include a graphical user interface (GUI) or other user interface to provide unidirectional or bidirectional communication, input, and/or transfers of information and/or commands between the user device 102 and the SVAAS 104. In other aspects, this may include any other suitable methods, systems and techniques of interaction between the user device 102 and the SVAAS 104.

In other aspects, user device 102 may be a user interface of SVAAS 104, e.g., a GUI presented by and/or on the SVAAS 104 via a display and/or touchscreen, which may allow a user to provide the surgical video data 310 to the SVAAS 104, for example by uploading a computer file to the SVAAS 104 via the internet, the cloud, a disk drive, another computer device such as a smartphone, or any other suitable means of providing the surgical video data 310 to the SVAAS 104. The user interface may provide other functionality and/or allow other commands and/or control between a user, user device 102 and/or the SVAAS 104.

In some aspects, the user may be prompted to select one or more options associated with the operation of the SVAAS 104 when providing the surgical video data 310, or at any other time. In an aspect, the options may be presented to the user via a GUI on the user device 102. In some aspects, the SVAAS 104 may present options to a user, which may be selection of one more: metrics to generate; performance assessments to generate; whether a surgical video data 310 should be edited; surgical phases to assess, whether a surgical video data 310 should be semantically segmented; board certifications; credentialing options; pay-per-performance options; recipients of data, information, metrics and/or assessments generated by the SVAAS 104; as well as any other suitable options associated with one or more operational parameters of the SVAAS 104. In other aspects, the SVAAS 104 may not require any selections from a user and may operate autonomously.

Block 604 of method 600 may include processing, by the one or more processors 120 of SVAAS 104, the surgical video data 310 using one or more trained assessment ML models, such as 150A, 150B, 150C, to generate one or more assessment metrics. In an aspect, the one or more assessment ML models 150A, 150B, 150C are trained by ML model training module 142 using historical ophthalmic surgery data, which may be stored in memory 122 and/or database 126.

Once trained, the one or more trained assessment ML models 150A, 150B, 150C may be loaded at runtime into ML operation module 144, process the surgical video data 310, and generate one or more assessment metrics which may include one or more of a surgical instrument metric, a surgical phase metric, and/or an AC metric. The SVAAS 104 may intelligently and/or autonomously select which of the one or more trained assessment ML models (e.g., 150A, 150B, 150C) to process the surgical video data 310. Additionally, or alternatively, in another aspect, the SVAAS 104 may make ML model selections semi-autonomously, e.g., based on minimal user input. In other aspects, the SVAAS 104 may make ML model selections only based on user input, e.g., based upon the metrics and/or performance assessments a user may select when providing the surgical video data 310 to the SVAAS 104.

In an aspect of block 604 of method 600, surgical instrument assessment ML model 150A may generate one or more surgical instrument metrics. The surgical instrument ML model 150A may be trained using the historical ophthalmic surgery data in some aspects, and/or in other aspects trained on only a portion or a subset of the historical ophthalmic surgery data which may be pertinent to a surgical instrument e.g., instrument subset data, either or both types of training data may be stored in memory 122 and/or database 126. The instrument subset data may include one or more images indicating (e.g., via labels) one or more of: the presence of a surgical instrument in an image (instrument presence); the identification of the surgical instrument in an image (instrument identification); the color of the surgical instrument (instrument color); and/or the material the instrument may be comprised of (instrument material), as well as any other suitable information which may assist an ML model in generating a surgical instrument metric. In another example, CAD data of a surgical instrument may be used as instrument subset data which may include 3D data providing rendering and pose of an instrument.

In an aspect, example surgical instrument metrics the trained surgical instrument assessment ML model 150A may generate may include identifying one or more instruments and the order in which each surgical instrument is used during a surgical procedure (instrument ordering); the location of one or more surgical instruments (instrument location) which may include identification of one or more instruments and the instrument's location with respect to another identified instrument, an identified landmark, the image frame, or any other suitable manner of identifying location/localization, to name but a few; and/or the duration of time an identified instrument is used during a phase and/or step in a procedure (instrument duration). Instruments which the SVAAS 104 may be able to detect based upon training may include one or more of a cystotome; a chopper/second instrument; an irrigation/aspiration handpiece; a keratome; a lens injector; a paracentesis blade; a phacoemulsification handpiece; utrata forceps, a stabilizer, a lens dialer and/or a cannula, however, other surgical instruments may also be trained and detected by a trained ML model, including those unrelated to ophthalmic surgery.

Referring back to block 604 of method 600, in another aspect trained surgical phase assessment ML model 150B may generate one or more surgical phase metrics. The surgical phase assessment ML model 150B may be trained using the historical ophthalmic surgery data in some aspects, and/or only a portion or a subset of the historical ophthalmic surgery data in other aspects which may be pertinent to a surgical phase, e.g., a surgical phase subset data. Surgical phase subset data may include one or more images indicating (e.g., labeled) one or more of: the step in a surgical procedure (surgical step identification); the phase of a surgical procedure (surgical phase identification); as well as any other suitable indications which may assist an ML model in generating a surgical phase metric. In an aspect, the trained surgical phase assessment ML model 150B may include an ensemble model consisting of a trained Densenet169 convolutional NN and an I3D model trained by combining the second to last layer output of each model as the input to two fully connected layers of size 128 and 32 nodes.

Again, referring back to block 604 of method 600, trained AC assessment ML model 150C may generate one or more AC metrics. The AC assessment ML model 150C may be trained using the historical ophthalmic surgery data in some aspects, and/or in other aspects only a portion or a subset of the historical ophthalmic surgery data which may be pertinent to an AC, e.g., a capsulotomy subset data which may include one or more images indicating (e.g., with labels) one or more of: identifying the capsulorrhexis (capsulorrhexis identification); identifying the limbus location (limbus location); identifying the purkinje image location (purkinje image location); identifying an anatomical landmark (anatomical landmark); and/or identifying an anatomical change (anatomical change), e.g., an anatomical change in the capsulotomy, as well as any other suitable indications which may assist an ML model in generating an AC metric. AC metrics the SVAAS 104 may generate using the trained AC assessment ML model 150C may include a capsulorrhexis size, a capsulorrhexis centration, a capsulorrhexis eccentricity, a capsulorrhexis circularity, a capsulorrhexis smoothness, or a fluidity of a rhexis formation. One or more of the AC metrics may be used by the SVAAS 104 to assess the performance of the surgeon.

Block 606 of method 600 may include generating, by the one or more processors 120 and based upon at least the one or more assessment metrics, a performance assessment of the surgeon. In some aspects, one or more of the surgical instrument metrics may be used by the SVAAS 104 to assess the performance of the surgeon. In some aspects, one or more of the surgical phase metrics may be used by the SVAAS 104 to assess the performance of the surgeon. In some aspects, one or more of the AC metrics may be used by the SVAAS 104 to assess the performance of the surgeon.

At block 608 the method 600 may include providing, by the one or more processors 120 to a user device 102, the performance assessment of the surgeon. In some aspects, this may include generating, by the one or more processors 120 and based upon at least the one or more assessment metrics, a performance assessment of the surgeon. In some examples, the performance assessment may include one or more of the following assessments: a skill level assessment; a phase of surgery duration assessment; a surgical quality assessment; a skill progression assessment; a board certification assessment; a credentialing assessment; a pay-for-performance assessment; and/or an early warning assessment. The one or more performance assessments may include qualitative or quantitative feedback; scoring; relative skill of the surgeon (e.g., trainee or expert). However, any form of assessment may be provided according to the systems, methods, and techniques of the present inventions.

The performance assessment may be provided asynchronously and/or at various times during and/or after a surgery, e.g., immediate feedback/in real-time, within a reasonable time frame soon after the surgery, long-term over days/months/years, as well as any other suitable time frame.

In an embodiment, method 600 may include the SVAAS 104 processing, by the one or more processors 120, the surgical video data 310 using a trained semantic segmentation ML model 150D to generate a semantic segmentation subset data 520 including one or more images indicating a semantic segmentation of an AC. The semantic segmentation ML model 150D may be trained using historical semantic segmentation data including at least one or more images of a classified AC. Classification may include identification, ground truth annotation, outlining and/or bound boxing one or more images and/or frames of anatomy or landmarks associated with an AC, e.g., a capsulorrhexis, the limbus, the purkinje image, as well as any other suitable anatomy or landmarks.

Method 600 may also include processing, by the one or more processors 120, the semantic segmentation subset data 520 and/or the surgical video data 310 using one or more trained assessment ML models (e.g., the AC assessment ML model 150C) to generate the AC metric discussed herein.

In an embodiment, the method 600 may include processing, by the one or more processors 120, the surgical video data 310 using a trained video editing ML model 150E to generate an edited surgical video data 420. The trained video editing ML model 150E may be trained using historical surgical phase activity data. In an aspect, the historical surgical phase activity data may include one or more images indicating one or more of paracentesis, a medication injection, a viscoelastic insertion, a main wound, a capsulorrhexis initiation, a capsulorrhexis completion, a hydrodissection, a phacoemulsification, a cortical removal, a lens insertion, a viscoelastic removal, or a wound closure.

Once the surgical video data 310 is processed by trained video editing ML model 150E, one or more images capturing phase inactivity are removed from the edited surgical video data 420 generated by trained video editing ML model 150E, which may include removing one or more images capturing phase inactivity by comparing temporal aspects and spatial aspects of the surgical video data 310, as well as any other suitable aspects of the surgical video data 310.

Although in some embodiments one or more trained ML models are contemplated as having specific functionality, in other embodiments systems and methods may include fewer and/or a single trained ML model(s), which may have the same functionality. Various combinations of trained ML models and associated functionality are contemplated as within the scope of the systems, methods and techniques disclosed.

Additionally, although cataract and/or ophthalmic surgery may be used as examples, any type of surgery may be contemplated by the present systems, methods and techniques.

### Additional Considerations

With the foregoing, users whose data is being collected and/or utilized may first opt-in. After a user provides affirmative consent, data may be collected from the user's device (e.g., a mobile computing device). In other embodiments, deployment and use of ML models at a client or user device may have the benefit of removing any concerns of privacy or anonymity, by removing the need to send any personal or private data to a remote server.

The following additional considerations apply to the foregoing discussion. Throughout this specification, plural instances may implement operations or structures described as a single instance. Although individual operations of one or more methods are illustrated and described as separate operations, one or more of the individual operations may be performed concurrently, and nothing requires that the operations be performed in the order illustrated. These and other variations, modifications, additions, and improvements fall within the scope of the subject matter herein.

The patent claims at the end of this patent application are not intended to be construed under 35 U.S.C. § 112(f) unless traditional means-plus-function language is expressly recited, such as “means for” or “step for” language being explicitly recited in the claim(s). The systems and methods described herein are directed to an improvement to computer functionality, and improve the functioning of conventional computers.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “one embodiment” or “an embodiment” means that a particular element, feature, structure, or characteristic described in connection with the embodiment is included in at least one embodiment. The appearances of the phrase “in one embodiment”, “in one aspect” or the like in various places in the specification are not necessarily all referring to the same embodiment.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the description. This description, and the claims that follow, should be read to include one or at least one and the singular also includes the plural unless it is obvious that it is meant otherwise.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Additionally, certain embodiments are described herein as including logic or a number of routines, subroutines, applications, or instructions. These may constitute either software (e.g., code embodied on a machine-readable medium) or hardware. In hardware, the routines, etc., are tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a field programmable gate array (FPGA) or an application-specific integrated circuit (ASIC) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

Hardware modules can provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connect the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory product to which it is communicatively coupled. A further hardware module may then, at a later time, access the memory product to retrieve and process the stored output. Hardware modules may also initiate communications with input or output products, and can operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method may be performed by one or more processors or processor-implemented hardware modules. The performance of certain of the operations may be distributed among the one or more processors, not only residing within a single machine, but deployed across a number of machines. In some example embodiments, the processor or processors may be located in a single location (e.g., within a building environment, an office environment or as a server farm), while in other embodiments the processors may be distributed across a number of locations.

The performance of certain of the operations may be distributed among the one or more processors, not only residing within a single machine, but deployed across a number of machines. In some example embodiments, the one or more processors or processor-implemented modules may be located in a single geographic location (e.g., within a building environment, an office environment, or a server farm). In other example embodiments, the one or more processors or processor-implemented modules may be distributed across a number of geographic locations.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

Upon reading this disclosure, those of skill in the art will appreciate still additional alternative structural and functional designs for the method and systems described herein through the principles disclosed herein. Thus, while particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended claims.

Thus, many modifications and variations may be made in the techniques, methods, and structures described and illustrated herein without departing from the spirit and scope of the present claims. Accordingly, it should be understood that the methods and apparatus described herein are illustrative only and are not limiting upon the scope of the claims.

What is claimed:

1. A computer-implemented method for using machine learning to analyze a surgical video and assess performance of a surgeon conducting a surgical procedure, comprising:
receiving, by one or more processors from a user device, surgical video data including one or more images capturing at least a portion of an ophthalmic surgical procedure;
processing, by the one or more processors, the surgical video data using one or more trained assessment machine learning models to generate one or more assessment metrics, wherein:
the one or more trained assessment machine learning models are trained using historical ophthalmic surgery data; and
the one or more assessment metrics include one or more of a surgical instrument metric, a surgical phase metric, or an anterior capsulotomy metric;
generating, by the one or more processors and based upon at least the one or more assessment metrics, a performance assessment of the surgeon; and
providing, by the one or more processors to the user device, the performance assessment of the surgeon.

2. The computer-implemented method of claim 1, wherein the historical ophthalmic surgery data includes one or more images indicating one or more of an instrument presence, an instrument identification, an instrument color, an instrument material, a surgical step identification, a surgical phase identification, a capsulorrhexis identification, a limbus identification, a pupil identification, a purkinje image identification, an anatomical landmark identification, or an anatomical change identification.

3. The computer-implemented method of claim 1, wherein the surgical instrument metric includes one or more of instrument ordering, instrument location, or instrument duration.

4. The computer-implemented method of claim 1, comprising:
generating, by the one or more processors, the surgical instrument metric using a trained surgical instrument assessment machine learning model, the trained surgical instrument assessment machine learning model trained using one or more of the historical ophthalmic surgery data or surgical instrument subset data, wherein the surgical instrument subset data includes one or more images indicating one or more of an instrument presence, an instrument identification, an instrument color, or an instrument material.

5. The computer-implemented method of claim 1, wherein the surgical phase metric includes one or more of a surgical step order or surgical step duration.

6. The computer-implemented method of claim 1, comprising:
generating, by the one or more processors, the surgical phase metric using a trained surgical phase assessment machine learning model, the trained surgical phase assessment machine learning model trained using one or more of the historical ophthalmic surgery data or phase subset data, wherein the phase subset data includes one or more images indicating one or more of a surgical step identification or a surgical phase identification.

7. The computer-implemented method of claim 1, wherein the anterior capsulotomy metric includes one or more of a capsulorrhexis size, a capsulorrhexis centration, a capsulorrhexis eccentricity, a capsulorrhexis circularity, a capsulorrhexis smoothness, or a fluidity of a rhexis formation.

8. The computer-implemented method of claim 1, comprising:
generating, by the one or more processors, the anterior capsulotomy metric using a trained anterior capsulotomy assessment machine learning model, the trained anterior capsulotomy assessment machine learning model trained using one or more of the historical ophthalmic surgery data or capsulotomy subset data, wherein the capsulotomy subset data includes one or more images indicating one or more of a capsulorrhexis identification, a limbus location, a purkinje image location, an anatomical landmark, or an anatomical change.

9. The computer-implemented method of claim 1, comprising:
processing, by the one or more processors, the surgical video data using a trained semantic segmentation machine learning model to generate a semantic segmentation subset data including one or more images indicating a semantic segmentation of a capsulorrhexis, the trained semantic segmentation machine learning model trained using historical semantic segmentation data including at least one or more images of a classified capsulorrhexis; and
processing, by the one or more processors, one or more of the semantic segmentation subset data or the surgical video data using the one or more trained assessment machine learning models to generate one or more assessment metrics.

10. The computer-implemented method of claim 1, wherein the performance assessment includes one or more of a skill level assessment, a phase of surgery duration assessment, a surgical quality assessment, a skill progression assessment, an anterior capsulotomy assessment, a board certification assessment, a credentialing assessment, a pay-for-performance assessment, or an early warning assessment.

11. The computer-implemented method of claim 1, comprising:
processing, by the one or more processors, the surgical video data using a trained video editing machine learning model to generate an edited surgical video data, wherein:
the trained video editing machine learning model is trained using historical surgical phase activity data, wherein the historical surgical phase activity data includes one or more images indicating one or more of a paracentesis, a medication injection, a viscoelastic insertion, a main wound, a capsulorrhexis initiation, a capsulorrhexis completion, a hydrodissection, a phacoemulsification, a cortical removal, a lens insertion, a viscoelastic removal, or a wound closure; and
the edited surgical video data removes one or more images capturing phase inactivity; and
one or more of:
providing, by the one or more processors, the edited surgical video data to the user device; or
processing, by the one or more processors, the edited surgical video data using one or more trained assessment machine learning models to generate one or more assessment metrics.

12. A computer system for using machine learning to analyze a surgical video and assess performance of a surgeon conducting a surgical procedure, comprising:
one or more processors; and
a memory comprising instructions, that when executed, cause the computer system to:
receive surgical video data including one or more images capturing at least a portion of an ophthalmic surgical procedure from a user device;
process the surgical video data using one or more trained assessment machine learning models to generate one or more assessment metrics, wherein:
the one or more trained assessment machine learning models are trained using historical ophthalmic surgery data; and
the one or more assessment metrics include one or more of a surgical instrument metric, a surgical phase metric, or an anterior capsulotomy metric;
generate a performance assessment of the surgeon based upon at least the one or more assessment metrics; and
provide the performance assessment of the surgeon to the user device.

13. The computer system of claim 12, wherein the historical ophthalmic surgery data includes one or more images indicating one or more of an instrument presence, an instrument identification, an instrument color, an instrument material, a surgical step identification, a surgical phase identification, a capsulorrhexis identification, a limbus identification, a pupil identification, a purkinje image identification, an anatomical landmark identification, or an anatomical change identification.

14. The computer system of claim 12, wherein:
the surgical instrument metric includes one or more of instrument ordering, instrument location, or instrument duration; and
the memory comprises further instructions that, when executed, cause the system to:
generate the surgical instrument metric using a trained surgical instrument assessment machine learning model, the trained surgical instrument assessment machine learning model trained using one or more of the historical ophthalmic surgery data or surgical instrument subset data, wherein the surgical instrument subset data includes one or more images indicating one or more of an instrument presence, an instrument identification, an instrument color, or an instrument material.

15. The computer system of claim 12, wherein:
the surgical phase metric includes one or more of a surgical step order or surgical step duration; and
the memory comprises further instructions that, when executed, cause the system to:
generate the surgical phase metric using a trained surgical phase assessment machine learning model, the trained surgical phase assessment machine learning model trained using one or more of the historical ophthalmic surgery data or phase subset data, wherein the phase subset data includes one or more images indicating one or more of a surgical step identification or a surgical phase identification.

16. The computer system of claim 12, wherein:
the anterior capsulotomy metric includes one or more of a capsulorrhexis size, a capsulorrhexis centration, a capsulorrhexis eccentricity, a capsulorrhexis circularity, a capsulorrhexis smoothness, or a fluidity of a rhexis formation; and
the memory comprising further instructions that, when executed, cause the system to:
generate the anterior capsulotomy metric using a trained anterior capsulotomy assessment machine learning model, the trained anterior capsulotomy assessment machine learning model trained using one or more of the historical ophthalmic surgery data or capsulotomy subset data, wherein the capsulotomy subset data includes one or more images indicating one or more of a capsulorrhexis identification, a limbus location, a purkinje image location, an anatomical landmark, or an anatomical change.

17. The computer system of claim 12, the memory comprising further instructions that, when executed, cause the system to:
process the surgical video data using a trained semantic segmentation machine learning model to generate a semantic segmentation subset data including one or more images indicating a semantic segmentation of a capsulorrhexis, the trained semantic segmentation machine learning model trained using historical semantic segmentation data including at least one or more images of a classified capsulorrhexis; and
process one or more of the semantic segmentation subset data or the surgical video data using the one or more trained assessment machine learning models to generate one or more assessment metrics.

18. The computer system of claim 12, wherein the performance assessment includes one or more of a skill level assessment, a phase of surgery duration assessment, a surgical quality assessment, a skill progression assessment, an anterior capsulotomy assessment, a board certification assessment, a credentialing assessment, a pay-for-performance assessment, or an early warning assessment.

19. The computer system of claim 12, the memory comprising further instructions that, when executed, cause the system to:
process the surgical video data using a trained video editing machine learning model to generate an edited surgical video data, wherein:
the trained video editing machine learning model is trained using historical surgical phase activity data, wherein the historical surgical phase activity data includes one or more images indicating one or more of a paracentesis, a medication injection, a viscoelastic insertion, a main wound, a capsulorrhexis initiation, a capsulorrhexis completion, a hydrodissection, a phacoemulsification, a cortical removal, a lens insertion, a viscoelastic removal, or a wound closure; and
the edited surgical video data removes one or more images capturing phase inactivity;
one or more of:
provide the edited surgical video data to the user device; or
process the edited surgical video data using one or more trained assessment machine learning models to generate one or more assessment metrics.

20. A non-transitory computer-readable storage medium storing executable instructions that, when executed by a processor, cause a computer to:
receive surgical video data including one or more images capturing at least a portion of an ophthalmic surgical procedure from a user device;
process the surgical video data using one or more trained assessment machine learning models to generate one or more assessment metrics, wherein:
the one or more trained assessment machine learning models are trained using historical ophthalmic surgery data; and
the one or more assessment metrics include one or more of a surgical instrument metric, a surgical phase metric, or an anterior capsulotomy metric;
generate a performance assessment of a surgeon based upon at least the one or more assessment metrics; and
provide the performance assessment of the surgeon to the user device.
